(19) United States
(12) Patent Application Publication
(10) Pub. No.: US 2018/0140522 A1
(43) Pub. Date: May 24, 2018

(54) USE OF CITRIC ACID ESTERS AS CORROSION INHIBITOR IN ALCOHOLIC COSMETIC COMPOSITIONS THAT CAN BE SPRAYED AS AN AEROSOL

(71) Applicant: Henkel AG & Co. KGaA, Duesseldorf (DE)

(72) Inventors: Thomas DOERING, Dormagen (DE); Gertraud TECKENBROCK, Sprockhoevel (DE)

(73) Assignee: Henkel AG & Co. KGaA, Duesseldorf (DE)

(21) Appl. No.: 15/570,390

(22) PCT Filed: Apr. 25, 2016
(86) PCT No.: PCT/EP2016/059190

§ 371 Date: Oct. 30, 2017

(30) Foreign Application Priority Data
Jun. 9, 2015 (DE) 10 2015 210 481.8

(51) Int. Cl. A61K 8/37; C07C 69/704; A61Q 15/00; A61K 8/04; A61K 8/55

(52) U.S. Cl. CPC A61K 8/37; C07C 69/704; A61K 2800/526; A61K 8/046; A61K 8/55; A61Q 15/00

(57) ABSTRACT

The present invention relates to the use of esters of citric acid as corrosion inhibitors in alcoholic cosmetic compositions containing water and electrolyte and are sprayable from metallic aerosol containers.

### CROSS-REFERENCE TO RELATED APPLICATION

This application is a U.S. National-Stage entry under 35 U.S.C. § 371 based on International Application No. PCT/EP2016/059190, filed Apr. 25, 2016 which was published under PCT Article 21(2) and which claims priority to German Application No. 10 2015 210 481.8, filed Jun. 9, 2015, which are all hereby incorporated in their entirety by reference.

### TECHNICAL FIELD

The present disclosure relates to the use of citric acid esters as corrosion inhibitors in alcoholic cosmetic compositions which contain at least one electrolyte, from about 0.02 to about 2.0 wt. % water and at least one propellant.

### BACKGROUND

Compositions containing propellants are dispensed into aerosol containers (also called spray cans). The compositions in these containers are sprayed as aerosols with the aid of a propellant. The use of aerosol containers for the spraying of cosmetic agents, such as deodorants, antiperspirants, hair-dyeing agents, hair sprays and hair styling agents offers many advantages. For example, the composition is distributed evenly on the application surface and it can be dispensed evenly and with good control. Furthermore, the consumption of the composition per application can be reduced so that packaging waste is reduced as a result of the longer usability.

Containers made of metal (aluminum, tinplate, tin), protected and/or shatterproof plastic or glass coated with plastic on the outside are usually used as pressure-tight containers for such aerosol containers. With use of containers made of plastic, glass or aluminum, the containers do not incur and damage due to corrosion, however, these containers are considerably more expensive to manufacture than commonly used containers made of tinplate or tin. Therefore, these containers are only used in very small quantities or are not used at all.

Containers made of steel and tin are used most due to the good commercial compatibility and affordable manufacturing costs. However, these containers are subject to undesired corrosion by certain ingredients of the cosmetic agents contained therein.

The corrosion occurring in these aerosol containers normally takes place in an electrochemical process, wherein electrons of the metal of the container and/or valve disk are transferred to electron acceptors in the cosmetic agent. In the process, the metal of the container is oxidized and destroyed in this manner. The use of electrolytes in combination with aqueous solutions, in particular, promotes corrosion of metallic aerosol containers, because electrolytes in aqueous solutions dissociate into ions and thereby cause electrical conductivity, which accelerates the electrochemical corrosion. The corrosion of these containers and/or valve disks has a contaminating effect on the cosmetic agent container therein and destroys the aerosol container and/or valve disk by forming perforations or cracks, which entails a diminished storage stability of filled aerosol containers.

In the prior art, metal aerosol containers coated with protective lacquers on the parts with which the cosmetic agents come into contact are used in order to prevent corrosion of these containers. However, such protective lacquers are complicated to apply and therefore increase the costs of the production process of aerosol containers and valves considerably. The metals which are used, particularly steel sheet, are provided with a thin tin coating, which has a higher corrosion barrier against the corrosive substances contained in the cosmetic agents, particularly chloride ions, acids and bases, than the untreated steel. However, there is the risk that the galvanizing is not applied absolutely consistently. In this case, so-called pitting can occur in defective areas.

The corrosion of containers made of tin and steel can also be avoided and/or prevented if cosmetic agents which are anhydrous and/or contain only anhydrous volatile organic solvents are used. However, due to statutory changes and customer wishes, the amount of volatile organic solvents (also called volatile organic compounds or VOC) must be reduced and replaced with water and/or alcohols, which usually contain residual amounts of water. However, the use of water or alcohols containing water increases the risk of corrosion of metallic aerosol containers.

In the prior art, therefore, corrosion inhibitors are frequently used with the use of cosmetic agents that contain water and are sprayable as an aerosol. Such inhibitors can be applied as a coating on the parts of the aerosol container coming into contact with the cosmetic composition or added to the cosmetic compositions.

Corrosion inhibitors known in the prior art include, for example, alkali metal nitrites and benzoates, borates, alkanolamines and amides, amine compounds such as morpholine, amides or also silicones. A disadvantage of the nitrites and borates is their irritating effect, which is why they are only conditionally suitable as a corrosion inhibitor for cosmetic agents which come into contact with the human skin. The known amines and amides have adequate cosmetic compatibility, but their corrosion-inhibiting effect is inadequate, so the corrosion of the aerosol container is not satisfactorily prevented in the long term.

Therefore, there is a requirement for corrosion inhibitors which have a highly corrosion-inhibiting effect, do not have negative influences on the chemical or aesthetic properties of the cosmetic compositions and are cosmetically and toxicologically harmless.

The task of the present disclosure was to provide a corrosion inhibitor which has a high corrosion-inhibiting effect and is therefore suitable for use in aqueous cosmetic agents containing electrolytes, which should be sprayed. In addition, this corrosion-inhibitor should have good cosmetic and toxicological compatibility and should not have any negative interactions with the normal ingredients of the cosmetic agents.

### BRIEF SUMMARY

An alcoholic cosmetic composition is provided herein. The cosmetic composition includes an ester of citric acid having the formula (I),

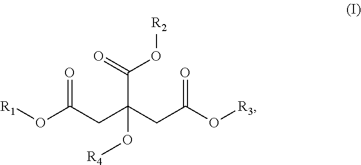

wherein

R₁ to R₃, independently of each other, denote a C₁-C₁₀-alkyl group, and

R₄ denotes hydrogen or a C₁-C₄-alkyl group. The cosmetic composition further includes at least one propellant, at least one electrolyte in a total amount of from about 0.01 to about 1.0 wt. %, and water in a total amount of from about 0.02 to about 2.0 wt. %. Each are relative to the total weight of the alcoholic cosmetic composition. The ester is utilized as a corrosion inhibitor in the alcoholic cosmetic composition.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it was found that the use of esters of citric acid in alcoholic cosmetic compositions which container water and at least one electrolyte and should be sprayed as an aerosol achieve an outstanding corrosion-inhibiting effect. Moreover, such esters are cosmetically and toxicologically harmless and do not result in negative interactions with other ingredients of cosmetic agents.

Therefore, the subject of the present disclosure is the use of an ester of citric acid having the formula (I)

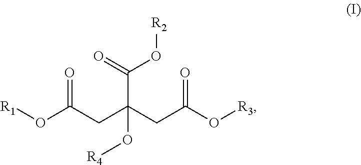

wherein
R₁ to R₃, independently of each other, denote a C₁-C₁₀-alkyl group, and
R₄ denotes hydrogen or a C₁-C₄-alkyl group, as a corrosion inhibitor in alcoholic cosmetic compositions which contain at least one propellant, at least one electrolyte in a total amount of from about 0.01 to about 1.0 wt. % and water in a total amount of from about 0.02 to about 2.0 wt. %, relative to the total weight of the alcoholic cosmetic composition in each case.

With the use of certain esters of citric acid, alcoholic cosmetic compositions can be formulated which can contain both water and electrolyte and are sprayable as an aerosol without the occurrence of corrosion of the aerosol container and/or valve disk. Moreover, these esters have outstanding cosmetic compatibility and are toxicologically harmless. Furthermore, their use does not result in negative interactions with other ingredients of the cosmetic agents, so it is not necessary to adapt already existing formulations.

As contemplated herein, the term “corrosion inhibitors” is understood to mean substances which impede corrosion of the metal container and/or valve disk when present in the cosmetic composition. The inhibition of corrosion can take place by impeding the electrochemical reaction and by means of physical or chemical interaction. In the physical interaction, absorption of the inhibitor takes place by electrostatic attraction or prevailing Van der Waals forces on the metal surface, whereas in the chemical interaction a protective layer is formed by a reaction of the inhibitor with the metal to be protected.

Furthermore, the term “alcoholic compositions” in the context of the present disclosure is understood to mean compositions which contain at least one monovalent C₁-C₄-alcohol, particularly ethanol, in an amount of at least 5 wt. %, relative to the total weight of the cosmetic composition. The at least one monovalent C₁-C₄-alcohol is preferably used as a solvent, particularly a cosmetically compatible carrier.

Moreover, the term “electrolyte” in the context of the present disclosure is understood to means substances which can release ions by means of dissociation in solution, particularly in an aqueous or aqueous-alcoholic solution. With the release of ions and the mobility, the solution forms an ion-conducting medium with electrical conductivity.

The term “fatty acid” as used in the scope of the present disclosure should be understood as aliphatic carboxylic acids having unbranched or branched carbon radicals with 4 to 40 carbon atoms. The fatty acids used in the scope of the present disclosure can be naturally occurring and synthetically manufactured fatty acids. Furthermore, the fatty acids can be mono- or polyunsaturated.

Finally, the term “fatty alcohol” in the scope of the present disclosure should be understood as, aliphatic, monovalent, primary alcohols having unbranched or branched hydrocarbon radicals with 4 to 40 carbon atoms. The fatty alcohols used in the scope of the present disclosure can also be mono- or polyunsaturated.

The specification of wt. % presently relates to the total weight of the cosmetic agent containing propellant, unless something different is indicated.

As contemplated herein, special esters of citric acid having the formula (I) are used as corrosion inhibitors for alcoholic cosmetic compositions. In this formula, the radicals R₁ to R₃ denote C₁-C₁₀-alkyl groups. Examples of such groups are methyl-, ethyl-, propyl-, isopropyl-, hydroxpropyl-, butyl-, sec-butyl-, isobutyl-, tert-butyl-, hydroxybutyl-, pentyl-, hexyl-, heptyl-, octyl-, nonyl- and decyl groups. The radical R₄ denotes C₁-C₄-alkyl groups, such as methyl-, ethyl-, propyl-, isopropyl-, hydroxypropyl-, butyl-, sec-butyl-, isobutyl-, tert-butyl-, and hydroxybutyl groups.

In the context of the present disclosure, preference is given to the use of esters of citric acid having the formula (I) in which the radicals R₁ to R₃ denote alkyl groups with a specific chain length. Therefore, preferred uses of the present disclosure are exemplified in that in the formula (I), the radicals R₁ to R₃, each independently of each other, denote a C₁-C₈-alkyl group, preferably a C₁-C₆-alkyl group, more preferably a C₁-C₄-alkyl group, particularly a C₂-alkyl group. The use of citric acid esters having the formula (I) in which the radicals R₁ to R₃ each denote a C₂-alkyl group, i.e. an ethyl group, has been shown to be particularly advantageous in regard to the corrosion-inhibiting effect of alcoholic cosmetic agents which contain water and at least one electrolyte.

As contemplated herein, preference is given to the use of citric acid esters having the formula (I) in which the radical R₄ denotes hydrogen. Preferred uses as contemplated herein are therefore exemplified in that the radical R₄ denotes hydrogen in the formula (I).

A particularly preferred embodiment of the present disclosure is therefore exemplified in that an ester of citric acid having the formula (Ia)

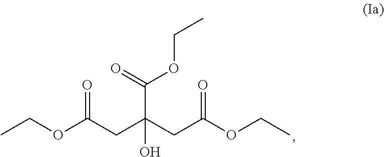 [SMILES: CCOC(=O)CC(O)(CC(=O)OCC)C(=O)OCC]

is used. Citric acid esters having the formula (Ia) are known by the INCI designation triethyl citrate and have the CAS number 77-93-0. With the use of triethyl citrate in alcoholic cosmetic compositions which contain water and at least one electrolyte, the corrosion of aerosol containers and/or valve disks containing metal can be completely prevented, in particular. Furthermore, triethyl citrate has good cosmetic compatibility and is completely toxicologically harmless. Moreover, the use of triethyl citrate does not cause undesired interactions with additional substances so that common formulations for compositions containing propellants can be added without further adjustment.

The at least one ester of citric acid of the formula (I) is preferably used in specific quantity ranges. Therefore, as contemplated herein, it is advantageous if the ester of citric acid having the formula (I) or the formula (Ia) is used in a total amount of from about 0.5 to about 5.0 wt. %, preferably from about 0.5 to about 4.0 wt. %, more preferably from about 0.5 to about 3.0 wt. %, particularly from about 1.0 to about 2.0 wt. %, relative to the total weight of the alcoholic cosmetic composition. The use of the aforementioned total amounts of citric acid esters having the formula (I) or (Ia) achieves good corrosion inhibition without causing incompatibilities with other ingredients of the cosmetic composition.

The cosmetic compositions used in the present disclosure are alcoholic cosmetic compositions. These compositions contain at least one C₁-C₄-alcohol. Therefore, preferred uses according to the present disclosure are exemplified in that the alcoholic cosmetic composition contains at least one monovalent C₁-C₄-alcohol selected from the group of methanol, ethanol, propanol, isopropanol, butanol and mixtures thereof, particularly ethanol, in a total amount of from about 15 to about 30 wt. %, preferably from about 18 to about 28 wt. %, particularly from about 20 to about 24 wt. %, relative to the total weight of the alcoholic cosmetic composition.

Furthermore, as contemplated herein it is preferred if the composition contains water in a total amount of from about 0.05 to about 1.8 wt. %, preferably from about 0.1 to about 1.6 wt. %, more preferably from about 0.2 to about 1.5 wt. %, particularly from about 0.5 to about 1.5 wt. %, relative to the total weight of the alcoholic composition. With use of citric acid esters of the formula (I) and/or (Ia), therefore, aqueous alcoholic solvents, particularly aqueous ethanol solvents are used in combination with electrolytes without corrosion of the metallic aerosol container and/or valve disk occurring. Aqueous alcoholic solvents, particularly aqueous ethanol, can be produced more affordably than anhydrous solvents and has good commercial availability.

The cosmetic agents also contain at least one electrolyte. Cosmetically compatible salts, acids and bases are preferably used as an electrolyte. Preferred uses as contemplated herein are therefore exemplified in that the at least one electrolyte is selected from the group of inorganic salts, in particular alkali metal salts; salts of di- and trivalent cations and n-alkylamidoalkyl-n, n-dialkyl-n-[hydroxyalkyl][ammonio]phosphates; organic salts, in particular n-octyl-1-[10-(4-octyliminopyridin-1-yl)decyl]pyridine-4-imine hydrochloride, 1-hexadecylpyridinium chlorides, silver citrates, zinc bis[(9Z,12R)-12-hydroxy-9-octadecenoate], potassium capryl glutamates, benzalkonium chlorides, benzethonium chlorides, 2-butyloctanoic acids, calcium magnesium silicates, hexadecyltrimethylammonium chlorides; inorganic and organic acids, inorganic and organic bases, in particular ammonia and amines, and mixtures thereof.

In this context, particularly preference is given to use of a compound having bacteria-inhibiting properties as an electrolyte and guarantees a deodorizing effect of the cosmetic agent. In this context, the term deodorizing effect is understood to mean the prevention of unpleasant odors, particularly under the armpits, which are caused by bacterial decomposition processes of the sweat, particularly underarm sweat. Therefore, it is particularly advantageous in this context if the at least one electrolyte is selected from the group of n-alkylamidoalkyl-n, n-dialkyl-n-[hydroxyalkyl]ammonio]phosphates having the formula (II)

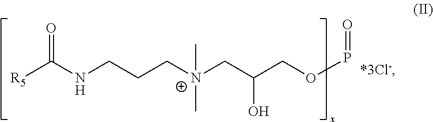

wherein
R₅ denotes a C₈-C₁₈-alkyl radical, particularly a cocoyl radical, n-octyl-1-[10-(4-octyliminopyridin-1-yl)decyl]pyridine-4-imine hydrochloride, 1-hexadecyl-pyridinium chlorides, silver citrates, zinc-bis[(9Z,12R)-12-hydroxy-9-octadecenoate] and mixtures thereof. The radical Rs in the formula (II) can preferably denote a cocoyl radical. This is understood to denote a mixture of C₈-C₁₈-carboxylic acid radicals, meaning the formula (II) is present as a non-uniform mixture with various carboxylic acid radicals with from about 8 to about 18 carbon atoms. Compounds of the formula (II) are known by the INCI designation cocamidopropyl PG-dimonium chloride phosphate and are commercially available, for example under the trade name Arlasilk PTC from the Croda company. The compound n-octyl-1-[10-(4-octyliminopyridin-1-yl)decyl]pyridin-4-imine hydrochloride is also referred to as octenidine HC₁ in the INCI nomenclature and has the CAS number 70775-75-6. The compound 1-hexadecyl pyridinium chloride having the CAS number 123 -03-5 is also referred to as cetylpyridinium chlorides according to the INCI nomenclature. The designation zinc bis [(9Z,12R)-12-hydroxy-9-octadecenoate] is understood to mean zinc ricinoleate according to the INCI nomenclature having the CAS number 13040-19-2. The aforementioned compounds dissociate in solution, particularly in alcoholic aqueous solution, in ions and can therefore accelerate or promote the corrosion of metallic aerosol containers and/or valve disks in aqueous solutions. With use of citric acid esters of the formula (I) and/or (Ia) as contemplated herein, however, these substances can be used without the risk of corrosion in alcoholic solutions containing water, particularly ethanol solutions containing water. In this manner, alcoholic solutions containing water which are sprayable as an aerosol can be formulated, which have an outstanding deodorant effect and only a small portion of or no volatile organic solvents (VOC).

The at least one electrolyte is used advantageous in specific quantity ranges. Uses as contemplated herein, therefore, are exemplified in that the cosmetic composition contains the at least one electrolyte in a total amount of from about 0.01 to about 0.3 wt. %, preferably from about 0.02 to about 0.2 wt. %, particularly from about 0.03 to about 0.1 wt. %, relative to the total weight of the alcoholic cosmetic composition. Use of the aforementioned total amounts of electrolyte, particularly an electrolyte having a deodorizing effect, ensures an adequate deodorizing effect by inhibiting bacteria which decomposes sweat into unpleasantly smelling compounds.

The cosmetic compositions used as contemplated herein are compositions that can be sprayed as an aerosol. Such compositions contain at least one propellant. The at least one propellant is preferably selected from the group of propane, propene, n-butane, isobutane, isobutene, n-pentane, pentene, isopentane, isopentene, methane, ethane, dimethylester, nitrogen, air, oxygen, nitrous oxide, 1,1,1,3-tetrafluorethane, heptafluoro-n-propane, perfluoroethane, monochlorodifluormethane, 1,1-difluorethane and tetrafluoropropenes and mixtures thereof, particularly propane and/or butane. As contemplated herein, butane means n-butane, iso-butane and mixtures of n-butane and iso-butane.

The at least one propellant is used advantageously in specific total amounts in order to achieve sufficient sprayability and size distribution of the cosmetic composition. In the context of the present disclosure, it is preferred that the cosmetic composition contains the at least one propellant in a total amount of from about 50 to about 95 wt. %, preferably from about 60 to about 85 wt. %, particularly from about 70 to about 80 wt. %, relative to the total weight of the alcoholic cosmetic compound.

A particularly preferred embodiment of the present disclosure is therefore characterized in at least one citric acid ester having the formula (Ia)

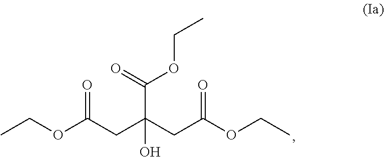 [SMILES: CCOC(=O)CC(O)(CC(=O)OCC)C(=O)OCC]

is used as a corrosion inhibitor in alcoholic cosmetic compositions which contain propane and/or butane in a total amount of from about 70 to about 80 wt. %, at least one electrolyte in a total amount of from about 0.03 to about 0.1 wt. % and water in a total amount of from about 0.5 to about 1.5 wt. %, relative to the total weight of the alcoholic cosmetic composition in each case.
wherein the at least one electrolyte is selected from the group of n-alkylamidoalkyl-n, n-dialkyl-n-[hydroxyalkyl]ammonio]phosphates having the formula (II)

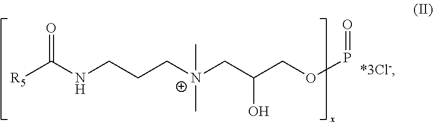

wherein
R₅ denotes a C₈-C₁₈-alkyl radical, particularly a cocoyl radical, n-octyl-1-[10-(4-octyliminopyridin-1-yl)decyl]pyridine-4-imine hydrochloride, 1-hexadecyl-pyridinium chlorides, silver citrates, zinc-bis[(9Z,12R)-12-hydroxy-9-octadecenoate] and mixtures thereof and
wherein the citric acid ester having the formula (Ia) is used in a total amount of from about 1.0 to about 2.0 wt. %, relative to the total weight of the alcoholic cosmetic composition.
With use of the citric acid ester having the formula (Ia), alcoholic aqueous cosmetic compositions which can be formulated with electrolytes can be sprayed as aerosols from aerosol containers containing metal without corrosion occurring due to the presence of water and electrolyte on the aerosol containers containing metal from the alcoholic cosmetic composition. Because the aforementioned electrolyte has an effective deodorizing effect, the formulation of deodorant composition containing water and which can be sprayed from metal-containing containers as an aerosol is possible.

Furthermore, in the context of the present disclosure, it can be provided that the alcoholic cosmetic composition contains a solvent differing from water and monovalent C₁-C₄-alcohol. Therefore, preferred inventive uses are characterized in that the alcoholic cosmetic composition additionally contains a solvent different from water, selected from the group of ethylene glycol, 1,2-propylene glycol, 1,3-propylene glycol, glycerin, n-butanol, 1,3-butylene glycol and mixtures thereof in a total amount of from about 15 to about 30 wt. %, preferably from about 18 to about 28 wt. %, particularly from about 20 to about 24 wt. %, relative to the total weight of the alcoholic cosmetic composition.

In order to further enhance the deodorizing effect, it can be advantageous as contemplated herein to also use an additional deodorant active ingredient for the at least one electrolyte with deodorizing effect. As contemplated herein, it is therefore preferred if the alcoholic cosmetic composition additionally comprises a deodorant active substance selected from the group of silver salts, aromatic alcohols, in particular 2-benzylheptan-l-ol and mixtures of 2-benzylheptan-1-ol and phenoxyethanol, 1,2-alkanediols having from about 5 to about 12 carbon atoms, in particular 3-(2-ethylhexyloxy)-1,2-propanediol, active substances against exoesterases, in particular against arylsulfatase, lipase, beta-glucuronidase and cystathionine beta-lyase, odor absorbers, in particular silicates, such as montmorillonite, kaolinite, illite, beidellite, nontronite, saponite, hectorite, bentonite, smectite and talcum, zeolites, zinc ricinoleate, cyclodextrins, deodorizing ion exchangers, germ-inhibiting agents, prebiotically effective components and mixtures thereof, in a total amount of from about 0.0001 to about 10 wt. %, preferably from about 0.001 to about 5.0 wt. %, more preferably from about 0.01 to about 2.5 wt. %, particularly from about 0.1 to about 1.0 wt. %, relative to the total weight of the alcoholic cosmetic composition.

The cosmetic composition can also contain at least one preservative in order to extend the stability and thus the usability of the alcoholic cosmetic composition. It can therefore be preferred if the alcoholic cosmetic composition additionally contains a preservative selected from the group of iodopropynylbutylcarbamates, parabens, phenoxyethanol, benzoic acid and salts thereof, dibromodicyanobutane, 2-bromo-2-nitro-propan-1,3-diol, imidazolidinylurea, 5-chloro-2-methyl-4-isothiazolin-3-on, 2-chloroacetamide, benzalkonium chloride, benzyl alcohol, salicylic acid and salicylates, 2-benzyl heptanol, 1,2-hexanediol, and mixtures thereof, in a total amount of from about 0.0001 to about 1.0 wt. %, preferably from about 0.001 to about 0.8 wt. %, more preferably from about 0.01 to about 0.5 wt. %, particularly from about 0.05 to about 0.3 wt. %, relative to the total weight of the alcoholic cosmetic composition.

In the context of the present disclosure, particular preference is given to the use of electrolyte having deodorizing properties. Therefore, it is advantageous if the alcoholic cosmetic composition is a deodorant composition which can be sprayed as an aerosol using propellant from aerosol containers, preferably aerosol containers containing metal. As contemplated herein, therefore particular preference is given to uses characterized in that the alcoholic cosmetic composition is a deodorant composition which is sprayable as an aerosol. In this context, an aerosol is understood to mean a dispersion of liquid droplets of the alcoholic cosmetic composition in the gaseous propellant.

Particularly preferred embodiments of the inventive use are listed in the following tables (all specifications in wt. %, unless otherwise indicated). These are deodorant compositions which are sprayable as aerosols.

| | Used esters of | Amount of ester of |
|---|---|---|
| No. | citric acid | citric acid used |
| E1 | Ester of the formula (I) | 0.5 to 5.0 |
| E2 | Ester of the formula (I) | 0.5 to 4.0 |
| E3 | Ester of the formula (I) | 0.5 to 3.0 |
| E4 | Ester of the formula (I) | 1.0 to 2.0 |
| E5 | Ester of the formula (Ia) | 0.5 to 5.0 |
| E6 | Ester of the formula (Ia) | 0.5 to 4.0 |
| E7 | Ester of the formula (Ia) | 0.5 to 3.0 |
| E8 | Ester of the formula (Ia) | 1.0 to 2.0 |

The citric acid esters listed under the above numbers E1 to E7 and their amounts are used as corrosion inhibitors in the following alcoholic cosmetic compositions K1 to K60, i.e. the citric acid esters listed under the numbers E1 and E2 and E3 and E4 and E5 and E6 and E7 and E8 are used in the corresponding amounts as a corrosion inhibitor in each of the cosmetic compositions K1 to K60.

| | K1 | K2 | K3 | K4 |
|---|---|---|---|---|
| Electrolyte | 0.01 to 1.0 | 0.01 to 0.3 | 0.02 to 0.2 | 0.03 to 0.1 |
| Water | 0.02 to 2.0 | 0.05 to 1.8 | 0.2 to 1.5 | 0.5 to 1.5 |
| C₁-C₄-alcohol | 15 to 30 | 16 to 29 | 18 to 28 | 20 to 24 |
| Propellant | Ad 100 | Ad 100 | Ad 100 | Ad 100 |
| | K5 | K6 | K7 | K8 |
| Electrolyte ¹⁾ | 0.01 to 1.0 | 0.01 to 0.3 | 0.02 to 0.2 | 0.03 to 0.1 |
| Water | 0.02 to 2.0 | 0.05 to 1.8 | 0.2 to 1.5 | 0.5 to 1.5 |
| C₁-C₄-alcohol | 15 to 30 | 16 to 29 | 18 to 28 | 20 to 24 |
| Propane and/or butane | Ad 100 | Ad 100 | Ad 100 | Ad 100 |
| | K9 | K10 | K11 | K12 |
| Electrolyte ¹⁾ | 0.01 to 1.0 | 0.01 to 0.3 | 0.02 to 0.2 | 0.03 to 0.1 |
| Water | 0.02 to 2.0 | 0.05 to 1.8 | 0.2 to 1.5 | 0.5 to 1.5 |
| C₁-C₄-alcohol | 15 to 30 | 16 to 29 | 18 to 28 | 20 to 24 |
| Propane and/or | Ad 100 | Ad 100 | Ad 100 | Ad 100 |
| butane (15:85) | | | | |
| | K13 | K14 | K15 | K16 |
| n-slkylamidoalkyl-n,n- | 0.01 to 1.0 | 0.01 to 0.3 | 0.02 to 0.2 | 0.03 to 0.1 |
| dialkyl-n-[hydroxy- | | | | |
| alkyl]ammonio]phos- | | | | |
| phates of the formula (II) | | | | |
| Water | 0.02 to 2.0 | 0.05 to 1.8 | 0.2 to 1.5 | 0.5 to 1.5 |
| C₁-C₄-alcohol | 15 to 30 | 16 to 29 | 18 to 28 | 20 to 24 |
| Propane and/or butane | Ad 100 | Ad 100 | Ad 100 | Ad 100 |
| | K17 | K18 | K19 | K20 |
| n-octyl-1-[10-(4- | 0.01 to 1.0 | 0.01 to 0.3 | 0.02 to 0.2 | 0.03 to 0.1 |
| octyliminopyridin-1- | | | | |
| yl)decyl]pyridin- | | | | |
| 4-imine hydrochloride | | | | |
| Water | 0.02 to 2.0 | 0.05 to 1.8 | 0.2 to 1.5 | 0.5 to 1.5 |
| C₁-C₄-alcohol | 15 to 30 | 16 to 29 | 18 to 28 | 20 to 24 |
| Propane and/or butane | Ad 100 | Ad 100 | Ad 100 | Ad 100 |
| | K21 | K22 | K23 | K24 |
| 1-hexadecyl-pyridinium | 0.01 to 1.0 | 0.01 to 0.3 | 0.02 to 0.2 | 0.03 to 0.1 |
| chlorides | | | | |
| Water | 0.02 to 2.0 | 0.05 to 1.8 | 0.2 to 1.5 | 0.5 to 1.5 |
| C₁-C₄-alcohol | 15 to 30 | 16 to 29 | 18 to 28 | 20 to 24 |
| Propane and/or butane | Ad 100 | Ad 100 | Ad 100 | Ad 100 |
| | K25 | K26 | K27 | K28 |
| Silver citrates | 0.01 to 1.0 | 0.01 to 0.3 | 0.02 to 0.2 | 0.03 to 0.1 |
| Water | 0.02 to 2.0 | 0.05 to 1.8 | 0.2 to 1.5 | 0.5 to 1.5 |
| C₁-C₄-alcohol | 15 to 30 | 16 to 29 | 18 to 28 | 20 to 24 |
| Propane and/or butane | Ad 100 | Ad 100 | Ad 100 | Ad 100 |
| | K29 | K30 | K31 | K32 |
| zink-bis[(9Z,12R)- | 0.01 to 1.0 | 0.01 to 0.3 | 0.02 to 0.2 | 0.03 to 0.1 |
| 12-hydroxy-9- | | | | |
| octadecenoate] | | | | |
| Water | 0.02 to 2.0 | 0.05 to 1.8 | 0.2 to 1.5 | 0.5 to 1.5 |
| C₁-C₄-alcohol | 15 to 30 | 16 to 29 | 18 to 28 | 20 to 24 |
| Propane and/or butane | Ad 100 | Ad 100 | Ad 100 | Ad 100 |
| | K33 | K34 | K35 | K36 |
| Electrolyte | 0.01 to 1.0 | 0.01 to 0.3 | 0.02 to 0.2 | 0.03 to 0.1 |
| Ethanol | 15 to 30 | 17 to 29 | 18 to 28 | 20 to 24 |
| Water | 0.02 to 2.0 | 0.05 to 1.8 | 0.2 to 1.5 | 0.5 to 1.5 |
| Propellant | Ad 100 | Ad 100 | Ad 100 | Ad 100 |
| | K37 | K38 | K39 | K40 |
| Electrolyte ¹⁾ | 0.01 to 1.0 | 0.01 to 0.3 | 0.02 to 0.2 | 0.03 to 0.1 |
| Ethanol | 15 to 30 | 17 to 29 | 18 to 28 | 20 to 24 |
| Water | 0.02 to 2.0 | 0.05 to 1.8 | 0.2 to 1.5 | 0.5 to 1.5 |
| Propellant | Ad 100 | Ad 100 | Ad 100 | Ad 100 |
| | K41 | K42 | K43 | K44 |
| n-slkylamidoalkyl-n,n- | 0.01 to 1.0 | 0.01 to 0.3 | 0.02 to 0.2 | 0.03 to 0.1 |
| dialkyl-n-[hydroxy- | | | | |
| alkyl]ammonio]phos- | | | | |
| phates of the formula (II) | | | | |
| Ethanol | 15 to 30 | 17 to 29 | 18 to 28 | 20 to 24 |
| Water | 0.02 to 2.0 | 0.05 to 1.8 | 0.2 to 1.5 | 0.5 to 1.5 |
| Propane and/or butane | Ad 100 | Ad 100 | Ad 100 | Ad 100 |
| | K45 | K46 | K47 | K48 |
| n-octyl-1-[10-(4- | 0.01 to 1.0 | 0.01 to 0.3 | 0.02 to 0.2 | 0.03 to 0.1 |
| octyliminopyridin-1- | | | | |
| yl)decyl]pyridin- | | | | |
| 4-imine hydrochloride | | | | |
| Ethanol | 15 to 30 | 17 to 29 | 18 to 28 | 20 to 24 |
| Water | 0.02 to 2.0 | 0.05 to 1.8 | 0.2 to 1.5 | 0.5 to 1.5 |
| Propane and/or butane | Ad 100 | Ad 100 | Ad 100 | Ad 100 |
| | K49 | K50 | K51 | K52 |
| 1-hexadecyl-pyridinium | 0.01 to 1.0 | 0.01 to 0.3 | 0.02 to 0.2 | 0.03 to 0.1 |
| chlorides | | | | |
| Ethanol | 15 to 30 | 17 to 29 | 18 to 28 | 20 to 24 |
| Water | 0.02 to 2.0 | 0.05 to 1.8 | 0.2 to 1.5 | 0.5 to 1.5 |
| Propane and/or butane | Ad 100 | Ad 100 | Ad 100 | Ad 100 |
| | K53 | K54 | K55 | K56 |
| Silver citrates | 0.01 to 1.0 | 0.01 to 0.3 | 0.02 to 0.2 | 0.03 to 0.1 |
| Ethanol | 15 to 30 | 17 to 29 | 18 to 28 | 20 to 24 |
| Water | 0.02 to 2.0 | 0.05 to 1.8 | 0.2 to 1.5 | 0.5 to 1.5 |
| Propane and/or butane | Ad 100 | Ad 100 | Ad 100 | Ad 100 |
| | K57 | K58 | K59 | K60 |
| zink-bis[(9Z,12R)- | 0.01 to 1.0 | 0.01 to 0.3 | 0.02 to 0.2 | 0.03 to 0.1 |
| 12-hydroxy-9- | | | | |
| octadecenoate] | | | | |
| Ethanol | 15 to 30 | 17 to 29 | 18 to 28 | 20 to 24 |
| Water | 0.02 to 2.0 | 0.05 to 1.8 | 0.2 to 1.5 | 0.5 to 1.5 |
| Propane and/or butane | Ad 100 | Ad 100 | Ad 100 | Ad 100 |

¹⁾ Electrolyte is selected from the group of n-slkylamidoalkyl-n,n-dialkyl-n-[hydroxyalkyl]ammonio]phosphates of the formula (II) above, n-octyl-1-[10-(4-octyliminopyridin-1-yl)decyl]pyridine-4-imine hydrochloride, 1-hexadecyl-pyridinium chlorides, silver citrates, zinc-bis[(9Z,12R)-12-hydroxy-9-octadecenoate] and mixtures thereof.

Use of the citric acid ester having the formula (I) and/or (Ia) in the aforementioned particularly preferred embodiments as contemplated herein achieves an outstanding corrosion inhibition. This makes it possible to use alcoholic solvents containing water, particularly ethanol containing water, in combination with deodorant active ingredients present as electrolyte in cosmetic compositions which can be sprayed as an aerosol from containers containing metal.

A second subject of the present disclosure is a method for corrosion inhibition of metallic aerosol containers and/or valve disks, wherein an alcoholic composition having at least one propellant, at least one electrolyte in total amount of from about 0.01 to about 1.0 wt. % and water in a total amount of from about 0.02 to about 2.0 wt. %, relative to the total weight of the alcoholic cosmetic composition in each case, are contained with an ester of citric acid having the formula (I)

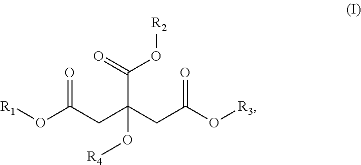

wherein
R₁ to R₃, independently of each other, denote a C₁-C₁₀-alkyl group, and
R₄ denotes hydrogen or a C₁-C₄-alkyl group, mixed and filled in an aerosol can which is them charged with a propellant and sealed by employing a valve with a valve disk.

The method as contemplated herein achieves a significantly improved inhibition of corrosion of aerosol containers and/or valve disks in comparison with methods in which no esters of citric acid have the formula (I) are used.

The following examples explain the present disclosure without limiting it.

### EXAMPLES

### 1. Corrosion Test

The following cosmetic compositions were produced, wherein preference is given to the use of esters having the formula (Ia) as citric acid esters of the formula (I) (all specifications in wt. %):

| Raw material | V | E* |
|---|---|---|
| Ethanol, 96% (contains 4 wt. % water) | 23.7 | 22.2 |
| Citric acid ester of the formula (I) | — | 1.50 |
| Electrolyte ¹⁾ | 0.075 | 0.075 |
| Preservative ²⁾ | 0.125 | 0.125 |
| Perfume | 1.0 | 1.0 |
| Propellant ³⁾ | Ad 100 | Ad 100 |

¹⁾ Arlasilk PTC (INCI designation: Cocamidopropyl PG-Dimomum Chloride Phosphate; 45-48 wt. % solution in water; Croda)
²⁾ Symdiol 68T (INCI designation: 1,2-Hexanediol, Caprylyl Glycol, Tropolone; Symrise)
³⁾ Propane/Butane in a ratio of 15:85
*as contemplated herein

Composition V contains 0.95 wt. % water, which arises from the use of ethanol containing water with a water portion of 4 wt. %. Composition E has a water portion of 0.89 wt. % based on the use of ethanol containing water.

Cosmetic compositions V (not as contemplated herein) and E (as contemplated herein) were filled in aluminum aerosol spray cans (Ball Aerocan) with a PPG7940 epoxy interior coating. A disk cover made of uncoated tinplate was used as a valve. The filled aerosol cans were then stored upright and upside down for 24 weeks each at 23° C. and 40° C.

Considerable corrosion appeared on the valve disks of the aerosol cans which did not contain the inventive composition V after 4 weeks at 40° C. and after 8 weeks at 23° C. By contrast, the aerosol filled with the inventive composition E did not show any corrosion of the valve disk even after 24 weeks of storage at 23° C. and 40° C. Therefore, the addition of the citric acid ester of the formula (I), particularly the formula (Ia), achieves effective corrosion protection of aerosol containers containing metal which are filled with aqueous cosmetic compounds containing electrolyte.

### 3. Sprayable Deodorant Formulations:

The citric acid ester of the formula (I) used in the following examples is preferably a citric acid of the formula (Ia):

| Raw material | 1. | 2 | 3 | 4 | 5 | 6 |
|---|---|---|---|---|---|---|
| Ethanol, 96% | 87.5 | 87.8 | 87.6 | 87.5 | 87.8 | 87.7 |
| Citric acid ester of the | 6.0 | 6.0 | 6.0 | 6.0 | 6.0 | 6.0 |
| formula (I) | | | | | | |
| Vitamin E acetate | 0.2 | 0.2 | 0.2 | 0.2 | 0.2 | 0.2 |
| Arlasilk PTC | 0.3 | 0.3 | 0.3 | 0.3 | 0.3 | 0.3 |
| Sensiva SC 50 | 0.5 | 0.5 | 0.5 | 0.5 | — | 0.3 |
| Symdiol 68 T | 0.5 | — | 0.2 | — | — | — |
| Tego Cosmo P 813 | — | 0.25 | 0.25 | — | — | — |
| SymDeo B125 | — | — | — | 0.5 | 0.5 | — |
| Dermosoft GMCY | — | — | — | — | 0.25 | — |
| Dermosoft DGMC | — | — | — | — | — | 0.2 |
| Melafresh T96-SC | — | — | — | — | — | 0.1 |
| Phenoxyethanol | 1.0 | 1.0 | 1.0 | 1.0 | 1.0 | 1.0 |
| Perfume | 4.0 | 4.0 | 4.0 | 4.0 | 4.0 | 4.0 |

Recipes 1 to 6 were dispensed in the weight ratio 1:3 with a mixture of propane and butane (propane/butane ratio=15/85) into aerosol cans containing metal. The following commercial products were used:

| | | Supplier/manu- |
|---|---|---|
| Commercial product | INCI | facturer |
| Arlasilk | Cocamidopropyl PG-dimonium | Croda |
| | chloride phosphate | |
| Dermosoft GMCY | Glyceryl Caprylate | Dr. Straetmans |
| Dermosoft DGMC | Polyglyceryl-2 Caprate | Dr. Straetmans |
| Melafresh T96-SC | Melaleuca Alternifolia | Southern Cross |
| | (Tea Tree) Leaf Oil | Botanicals P/L |
| Sensiva SC 50 | ETHYLHEXYLOXY- | Schülke & Mayr |
| | GLYCERIN | |
| SymDeo B125 | 2-methyl 5-cyclohexylpentanol | Symrise |
| Symdiol 68 T | 1,2-hexanediol, caprylyl glycol, | Symrise |
| | tropolone | |
| Tego Cosmo P 813 | POLYGLYCERYL-3 | Evonik |
| | CAPRYLATE | |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An ester of citric acid having the formula (I) 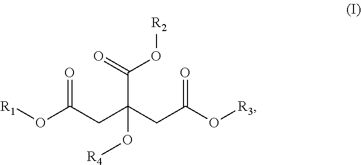
wherein
R₁ to R₃, independently of each other, denote a C₁-C₁₀-alkyl group, and
R₄ denotes hydrogen or a C₁-C₄-alkyl group,
wherein the ester is utilized as a corrosion inhibitor in alcoholic cosmetic compositions which contain comprises at least one propellant, at least one electrolyte in a total amount of from about 0.01 to about 1.0 wt. % and water in a total amount of from about 0.02 to about 2.0 wt. %, relative to the total weight of the alcoholic cosmetic composition in each case.

2. The ester according to claim 1, wherein in the formula (I), the radicals R₁ to R₃, each independently of each other, denote a C₁-C₈-alkyl group.

3. The ester according to claim 1, wherein the radical R₄ denotes hydrogen in the formula (I).

4. The ester according to claim 1, wherein the ester is an ester of citric acid having the formula (Ia) 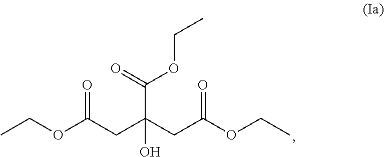 [SMILES: CCOC(=O)CC(O)(CC(=O)OCC)C(=O)OCC]
is utilized.

5. The ester according to claim 1, wherein the ester of citric acid having the formula (I) or the formula (Ia) is utilized in a total amount of from about 0.5 to about 5.0 wt. %, relative to the total weight of the alcoholic cosmetic composition.

6. The ester according to claim 1, wherein the alcoholic cosmetic composition comprises at least one monovalent C₁-C₄-alcohol selected from the group of methanol, ethanol, propanol, isopropanol, butanol and mixtures thereof, in a total amount of from about 15 to about 30 wt. %, relative to the total weight of the alcoholic cosmetic composition.

7. The ester according to claim 1 wherein the composition comprises water in a total amount of from about 0.05 to about 1.8 wt. %, relative to the total weight of the alcoholic composition.

8. The ester according to claim 1 wherein the at least one electrolyte is selected from the group of inorganic salts; salts of di-and trivalent cations and n-alkylamidoalkyl-n, n-dialkyl-n-[hydroxyalkyl][ammonio]phosphates; organic salts; inorganic and organic acids, inorganic and organic bases, and mixtures thereof.

9. The ester according to claim 1, wherein the at least one electrolyte is selected from the group of n-alkylamidoalkyl-n, n-dialkyl-n-[hydroxyalkyl]ammonio]phosphates having the formula (II) 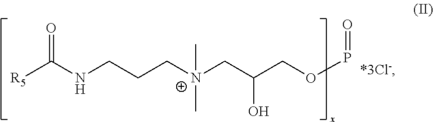
wherein
R₅ denotes a C₈-C₁₈-alkyl radical, particularly a cocoyl radical,
n-octyl-1-[10-(4-octyliminopyridin-1-yl)decyl]pyridine-4-imine hydrochloride, 1-hexadecyl-pyridinium chlorides, silver citrates, zinc-bis[(9Z,12R)-12-hydroxy-9-octadecenoate] and mixtures thereof.

10. The ester according to claim 1, wherein the cosmetic composition comprises the at least one electrolyte in a total amount of from about 0.01 to about 0.3 wt. %, relative to the total weight of the alcoholic cosmetic composition.

11. The ester according to claim 1, wherein the cosmetic composition comprises the at least one propellant in a total amount of from about 50 to about 95 wt. %, relative to the total weight of the alcoholic cosmetic compound.

12. The ester according to claim 1, wherein at least one citric acid ester having the formula (Ia) 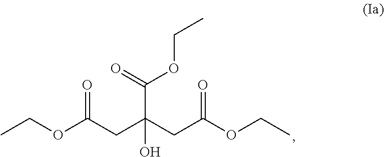 [SMILES: CCOC(=O)CC(O)(CC(=O)OCC)C(=O)OCC]
is utilized as a corrosion inhibitor in alcoholic cosmetic compositions which comprises propane and/or butane in a total amount of from about 70 to about 80 wt. %, at least one electrolyte in a total amount of from about 0.03 to about 0.1 wt. % and water in a total amount of from about 0.5 to about 1.5 wt. %, relative to the total weight of the alcoholic cosmetic composition in each case;
wherein the at least one electrolyte is selected from the group of n-alkylamidoalkyl-n, n-dialkyl-n-[hydroxyalkyl]ammonio]phosphates having the formula (II) 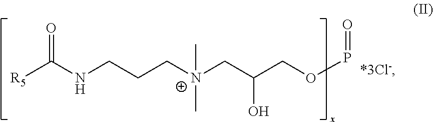
wherein
R₅ denotes a C₈-C₁₈-alkyl radical, particularly a cocoyl radical, n-octyl-1-[10-(4-octyliminopyridin-1-yl)decyl]pyridine-4-imine hydrochloride, 1-hexadecyl-pyridinium chlorides, silver citrates, zinc-bis[(9Z,12R)-12-hydroxy-9-octadecenoate] and mixtures thereof, and
wherein the citric acid ester having the formula (Ia) is utilized in a total amount of from about 1.0 to about 2.0 wt. %, relative to the total weight of the alcoholic cosmetic composition.

13. The ester according to claim 1, wherein the alcoholic cosmetic composition additionally comprises a solvent different from water, selected from the group of ethylene glycol, 1,2-propylene glycol, 1,3-propylene glycol, glycerin, n-butanol, 1,3-butylene glycol and mixtures thereof in a total amount of from about 15 to about 30 wt. %, relative to the total weight of the alcoholic cosmetic composition.

14. The ester according to claim 1, wherein the alcoholic cosmetic composition is a deodorant composition which is sprayable as an aerosol.

15. Method for corrosion inhibition of metallic aerosol containers and/or valve disks, wherein an alcoholic composition having at least one propellant, at least one electrolyte in total amount of from about 0.01 to about 1.0 wt. % and water in a total amount of from about 0.02 to about 2.0 wt. %, relative to the total weight of the alcoholic cosmetic composition in each case, are included with an ester of citric acid having the formula (I) 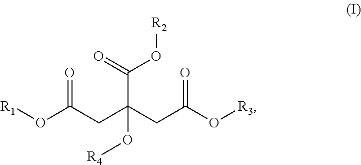
wherein
R₁ to R₃, independently of each other, denote a C₁-C₁₀-alkyl group, and R₄ denotes hydrogen or a C₁-C₄-alkyl group,
mixed and filled in an aerosol can which is then charged with a propellant and sealed by employing a valve with a valve disk.

16. An alcoholic cosmetic composition, comprising:
an ester of citric acid having the formula (I), 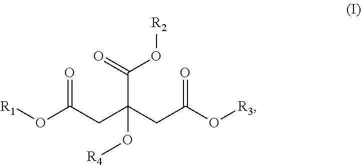
wherein
R₁ to R₃, independently of each other, denote a C₁-C₁₀-alkyl group, and
R₄ denotes hydrogen or a C₁-C₄-alkyl group;
at least one propellant;
at least one electrolyte in a total amount of from about 0.01 to about 1.0 wt. %; and
water in a total amount of from about 0.02 to about 2.0 wt. %;
wherein each are relative to the total weight of the alcoholic cosmetic composition; and
wherein the ester is utilized as a corrosion inhibitor in the alcoholic cosmetic composition.
